(19) United States
(12) Patent Application Publication
(10) Pub. No.: US 2018/0177988 A1
(43) Pub. Date: Jun. 28, 2018

(54) Medical Tube

(71) Applicant: TERUMO KABUSHIKI KAISHA, Kanagawa (JP)

(72) Inventors: Naoki Aramaki, Kanagawa (JP); Naotaka Chino, Kanagawa (JP); Hiroyuki Ishihara, Kanagawa (JP); Yuuki Souma, Kanagawa (JP)

(21) Appl. No.: 15/693,941

(22) Filed: Sep. 1, 2017

(30) Foreign Application Priority Data
Sep. 2, 2016 (JP) 2016-172068

(51) Int. Cl. A61M 27/00; A61M 25/00; A61M 25/04; A61B 17/11

(52) U.S. Cl. CPC A61M 27/008; A61B 17/1114; A61M 25/04; A61M 25/003

(57) ABSTRACT

A medical tube 10 includes a main shaft tube 20 that internally has a main shaft lumen 21, and a plurality of branch tubes 30 connected to each other at an intermediate position in an axial direction of the main shaft tube 20. The branch tube 30 has a branch lumen 31 through which pancreatic juice flows into the main shaft lumen 21. In a state where the main shaft tube 20 is inserted into a pancreas 100, a distal end of the branch tube 30 is disposed at a position separated outward from the main shaft tube 20 in a radial direction between the pancreas 100 and a jejunum 200.

### TECHNICAL FIELD

The present invention relates to a medical tube used for draining a fluid outward from the inside of a living body while indwelling the inside of the living body, for example.

### BACKGROUND

After surgery, to drain a fluid (liquid or gas generated by biological tissues or used for treatment) outward from the inside of a body, a medical drain tube (medical tube) as disclosed in Japanese Patent Application No. 4715504 is used.

As an example, in the pancreaticoduodenectomy, the medical drain tube is used for an anastomosis portion where the pancreas (pancreatic body and pancreatic tail) having the excised pancreatic head and the jejunum are anastomosed with each other. A trunk portion of the medical drain tube is caused to indwell after passing through the jejunum, and a distal portion of the medical drain tube is inserted into and caused to indwell the pancreas (main pancreatic duct), which is anastomosed with the jejunum. In this manner, the pancreatic fluid generated inside the pancreas is drained out of the body through the medical drain tube from the main pancreatic duct.

### SUMMARY

### Problem Solved

Incidentally, in a case where a plurality of biological organs is anastomosed with (connected to) each other by performing a surgery, there is a possibility that the fluid generated by the biological organs may leak to a boundary portion between the biological organs. For example, in the anastomosis portion where the pancreas and the jejunum are anastomosed with each other by means of the pancreaticoduodenectomy, the pancreatic juice leaks out from the pancreas side. If the pancreatic juice further leaks from the anastomosis portion into the abdominal cavity, the enzyme contained in the liquid may be activated, in some cases. In this case, there is a possibility that a patient may be adversely affected after the surgery.

The embodiments herein to solve the above-described problem, and an object is to provide a medical tube which can satisfactorily drain not only a fluid contained inside a biological organ, but also a fluid contained in a boundary portion which connects a plurality of biological organs to each other.

### Problem Solution

To achieve the above-described object, there is provided a medical tube including an elongated main shaft tube that internally has a main shaft lumen, and at least one branch extending portion that extends from an intermediate position in an axial direction of the main shaft tube. The branch extending portion has a flow path which communicates with the main shaft lumen from the outside of the main shaft tube. The main shaft tube can be inserted into a first biological organ. The branch extending portion is insertable into a portion between the first biological organ and a second biological organ connected to the first biological organ.

According to the above-described configuration, in the medical tube, the branch extending portion interlocking with the main shaft tube is disposed between the first and second biological organs. In this manner, the medical tube can collect a fluid contained therebetween. That is, the fluid contained in a boundary portion flows into the main shaft lumen of the main shaft tube through the flow path of the branch extending portion. Accordingly, the medical tube can cause the main shaft tube inserted into the biological organ to drain the fluid, and can also cause the branch extending portion to satisfactorily drain the fluid contained in the boundary portion which connects the plurality of biological organs to each other. As a result, it is possible to encourage recovery of a patient by suppressing the inconvenience that the fluid may flow out into the patient's body, for example, the peritoneal cavity.

In this case, the flow path can be a branch lumen which is disposed inside the branch extending portion, and through which an opening disposed in a distal end or on a side surface of the branch extending portion communicates with the main shaft lumen.

In this manner, the branch extending portion causes the fluid contained in the boundary portion of the plurality of biological organs to flow into the branch lumen from the opening disposed in the distal end or on the side surface. Accordingly, the fluid can smoothly flow to the main shaft lumen.

Alternatively, the flow path may be a groove portion which opens a side surface of the branch extending portion, and which extends along the axial direction of the branch extending portion.

In this manner, the branch extending portion causes the fluid contained in the boundary portion of the plurality of biological organs to flow into the groove portion from any position in the axial direction of the branch extending portion. Accordingly, the fluid can smoothly flow to the main shaft lumen.

In addition, the branch extending portion may be configured to be more flexible than the main shaft tube.

In this way, the branch extending portion is more flexible than the main shaft tube. Accordingly, the medical tube can indwell by aligning the branch extending portion with the shape of the boundary portion between the first and second biological organs. In addition, when the medical tube is removed, the branch extending portion moves along an arrangement position of the branch extending portion which is a non-adhesion location. Accordingly, the branch extending portion moves close to the main shaft tube while suppressing damage to an adhesion location of the first biological organ and the second biological organ. Accordingly, the branch extending portion moves integrally with the main shaft tube, and can smoothly withdraw from the boundary portion.

Furthermore, an outer diameter of the branch extending portion can be smaller than an outer diameter of the main shaft tube.

In this way, the outer diameter of the branch extending portion is smaller than the outer diameter of the main shaft tube. Accordingly, a gap for disposing the branch extending portion is reduced, and the adhesion between the biological organs is promoted. In addition, when the medical tube is removed, the branch extending portion can be easily pulled out from the boundary portion.

Furthermore, the branch extending portion can be shaped so as to face outward in a radial direction of the main shaft tube, and toward a distal side of the main shaft tube.

In this manner, when the branch extending portion is disposed in the boundary portion, the distal end of the branch extending portion can be simply disposed at a position separated from the main shaft tube. In addition, when the medical tube is removed, the branch extending portion can be pulled out from the boundary portion by causing the branch extending portion to smoothly move close to the main shaft tube.

Furthermore, a plurality of the branch extending portions may interlock with each other at the same position in the axial direction of the main shaft tube, and may extend radially outward in the radial direction of the main shaft tube.

In this way, the plurality of branch extending portions extend radially outward in the radial direction of the main shaft tube. Accordingly, the distal ends of the plurality of branch extending portions can be disposed in the vicinity of the peripheral edge of the boundary portion so as to be separated from each other in the circumferential direction. Accordingly, the medical tube can more reliably and effectively collect the fluid contained in the boundary portion.

In addition to the above-described configurations, in the plurality of the branch extending portions, the branch extending portions adjacent to each other may have mutually different axial lengths.

In this way, the plurality of branch extending portions adjacent to each other have mutually different axial lengths. Accordingly, the medical tube can collect the fluid by disposing the distal end of the branch extending portion at a position relatively close to the main shaft tube of the boundary portion.

Here, a distal portion of the branch extending portion may include distal fixing means for fixing the branch extending portion to the second biological organ.

In this way, the distal portion of the branch extending portion includes the distal fixing means. Accordingly, the distal portion of the branch extending portion can be prevented from slipping out of the boundary portion, and the fluid contained in the boundary portion can be more satisfactorily collected.

Furthermore, the distal end of the branch extending portion may have a distal opening support portion which can support the first or second biological organ. The distal opening support portion may have a forward opening portion which communicates with the branch lumen, and which is open in a distal end direction of the branch extending portion, and a lateral opening portion which communicates with the branch lumen, and which is open in a lateral portion of the branch extending portion.

In this way, the distal end of the branch extending portion has the distal opening support portion. Accordingly, while the first or second biological organ is supported, the fluid contained in a boundary portion can be satisfactorily collected from the forward opening portion and the lateral opening portion.

### Advantage of the Embodiments

According to the embodiments presented herein, a medical tube can satisfactorily drain not only a fluid contained inside a biological organ, but also a fluid contained in a boundary portion which connects a plurality of biological organs to each other.

### BRIEF DESCRIPTION OF THE DRAWINGS

FIG. 1 is a side view illustrating an overall configuration of a medical tube in accordance with embodiments of the present disclosure;

FIG. 2A is a first description-purpose view illustrating a flow in the pancreaticoduodenectomy in accordance with embodiments of the present disclosure;

FIG. 2B is a second description-purpose view illustrating a reconstructed state of an organ in the pancreaticoduodenectomy in accordance with embodiments of the present disclosure;

FIG. 3A is a side cross-sectional view illustrating an enlarged distal side of the medical tube in FIG. 1 in accordance with embodiments of the present disclosure;

FIG. 3B is a cross-sectional view obtained by cutting the medical tube in FIG. 1 along a shaft center of a branch tube in accordance with embodiments of the present disclosure;

FIG. 4 is a side cross-sectional view illustrating an enlarged state where the medical tube is applied to an anastomosis portion between the pancreas and the jejunum in accordance with embodiments of the present disclosure;

FIG. 5 is a side cross-sectional view illustrating an enlarged operation state when the medical tube is removed from the anastomosis portion in accordance with embodiments of the present disclosure;

FIG. 6 is a perspective view illustrating an enlarged distal side of a medical tube in accordance with embodiments of the present disclosure;

FIG. 7A is a schematic cross-sectional view illustrating a distal side of a medical tube in accordance with embodiments of the present disclosure;

FIG. 7B is a schematic cross-sectional view illustrating a distal side of a medical tube in accordance with embodiments of the present disclosure;

FIG. 7C is a schematic cross-sectional view illustrating a distal side of a medical tube in accordance with embodiments of the present disclosure;

FIG. 7D is a schematic cross-sectional view illustrating a distal side of a medical tube in accordance with embodiments of the present disclosure;

FIG. 8 is a side cross-sectional view illustrating an enlarged example of using a medical tube in accordance with embodiments of the present disclosure;

FIG. 9 is a side cross-sectional view illustrating an enlarged example of using a medical tube in accordance with embodiments of the present disclosure;

FIG. 10A is a partial perspective view illustrating a distal opening support portion of the medical tube in FIG. 9 in accordance with embodiments of the present disclosure;

FIG. 10B is a partial perspective view illustrating another distal opening support portion in accordance with embodiments of the present disclosure;

FIG. 10C is a partial perspective view illustrating yet another distal opening support portion in accordance with embodiments of the present disclosure.

### DETAILED DESCRIPTION

Hereinafter, a medical tube according will be described in detail with reference to embodiments and the accompanying drawings.

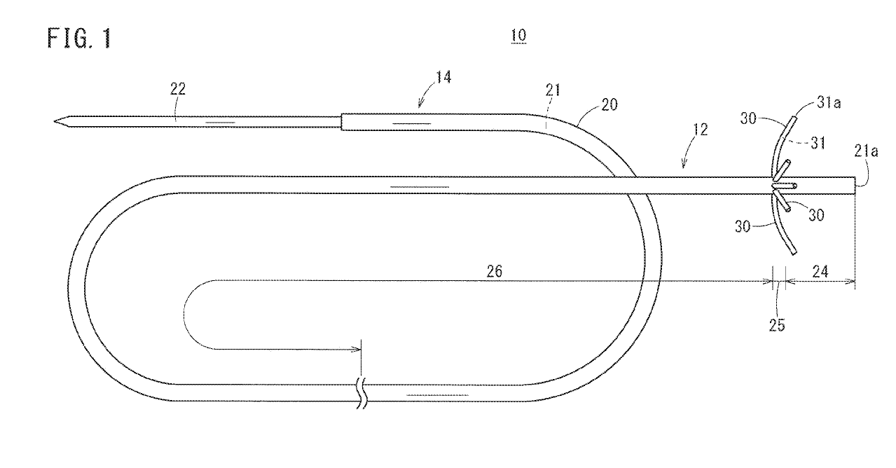

A medical tube 10 in accordance with embodiments of the present disclosure is formed in a hollow tube as illustrated in FIG. 1, and is configured to serve as a medical device through which a fluid (liquid or gas) can flow. In particular, the medical tube 10 is used as a drainage tube in which a distal portion side is caused to indwell a connection section of two biological organs inside a living body so that the fluid generated by (exuded from or secreted by) the biological organ flows and is drained to a proximal portion side exposed outward from the body. Accordingly, hereinafter, the medical tube 10 is referred to as a drain tube 10.

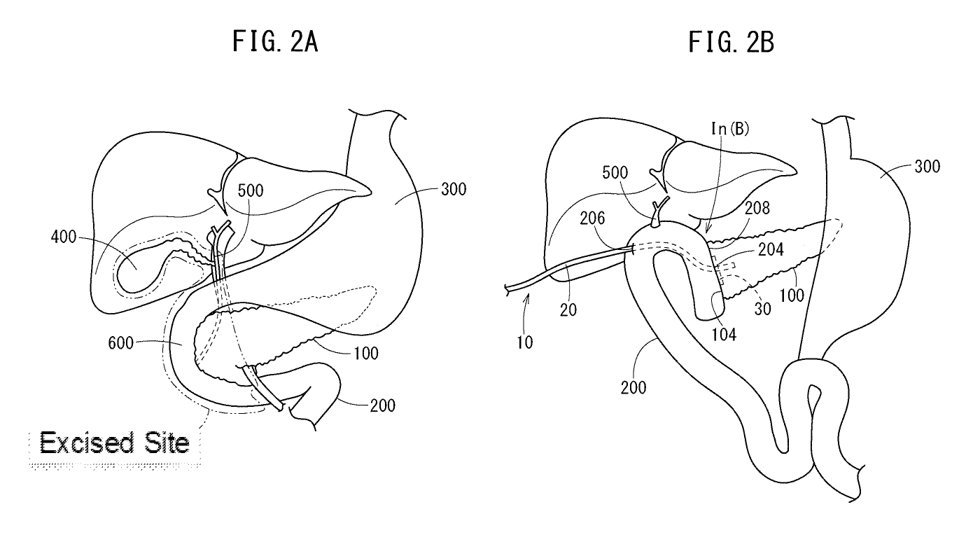

For example, as illustrated in FIGS. 2A and 2B, the drain tube 10 is applicable to a reconstructive surgery of an organ in the pancreaticoduodenectomy (including the pylorus-preserving pancreaticoduodenectomy). The pancreaticoduodenectomy is a surgery for cutting off a pancreatic head side of a pancreas 100, a portion of a stomach 300, a gall bladder 400, a bile duct 500, and a duodenum 600 to treat symptoms such as the pancreatic head cancer, the chronic pancreatitis, the bile duct cancer, the duodenal cancer, and the duodenal papillary carcinoma. As the reconstructive surgery, the end side pancreaticojejunostomy is performed. When the end side pancreaticojejunostomy is performed, in the drain tube 10, a distal portion side is caused to indwell the inside of the pancreas 100 which is a first biological organ, and a trunk portion is exposed outward from the body after passing through the inside of the jejunum 200 which is a second biological organ. In this manner, a pancreatic juice (fluid) generated from the pancreas 100 after surgery is drained outward from the body.

The pancreatic juice contains an enzyme (amylase) which decomposes proteins, fats and saccharides. In a case where the pancreatic juice leaks into an abdominal cavity, there is a possibility that the activated enzyme may cause serious complications (pancreatic fistula: for example, intraperitoneal hemorrhage, sepsis, peritonitis, or abdominal abscess). The drain tube 10 drains the pancreatic juice generated from the reconstructed pancreas 100 outward from the body, and restrains the leakage of the pancreatic juice into the abdominal cavity. In particular, the drain tube 10 can function to cause the pancreatic juice leaking out to a boundary portion B of an anastomosis portion In(B) (connection section) between of the pancreas 100 and the jejunum 200 to be drained outward from the body. Hereinafter, a configuration of the drain tube 10 will be described in detail.

As illustrated in FIG. 1, the drain tube 10 includes an insertion planned region 12 to be inserted into the body and an exposure planned region 14 exposed outward from the body, which are consecutively disposed in the axial direction. The total length (axial length) of the drain tube 10 is not particularly limited. However, for example, the length is 50 mm to 1,000 mm.

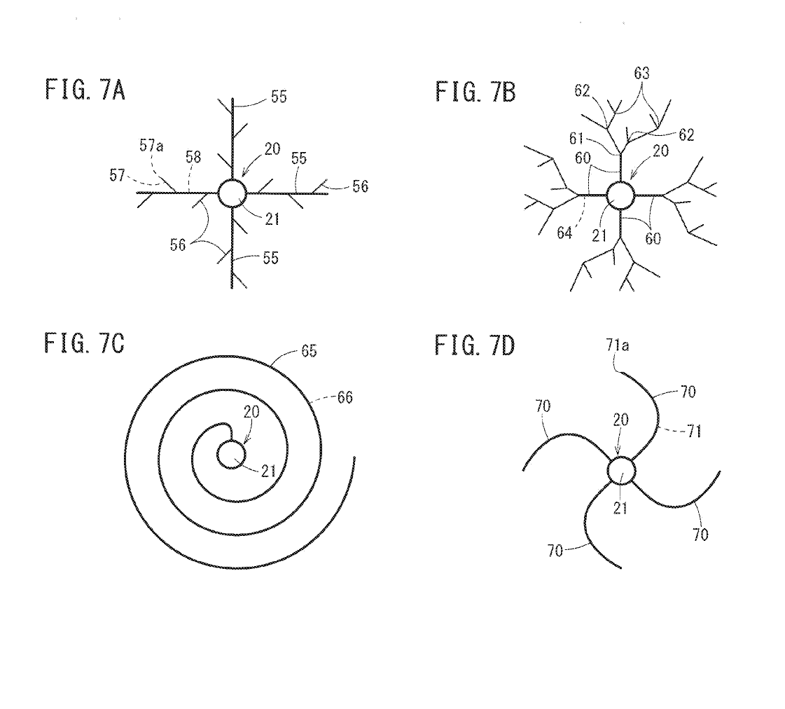

Specifically, the drain tube 10 has an elongate main shaft tube 20, which configures a main drain path for draining the fluid, and a plurality of branch tube 30 (branch extending portions) disposed at an intermediate position (in the vicinity of the distal portion of the insertion planned region 12) in the axial direction of the main shaft tube 20. Only one branch tube 30 may be disposed for the main shaft tube 20 (also refer to FIG. 7C).

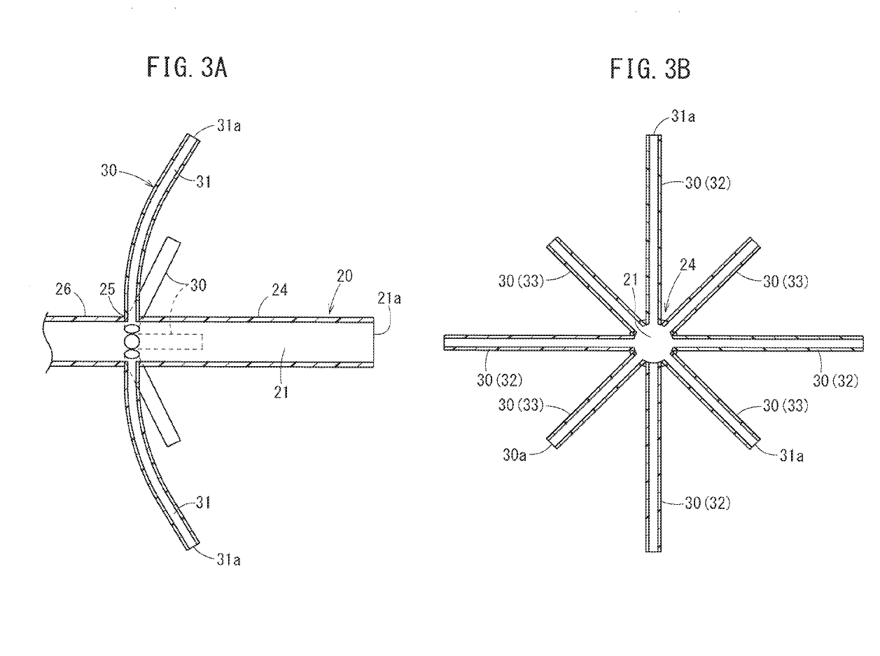

As illustrated in FIGS. 1, 3A, and 3B, a shaft center portion of the main shaft tube 20 has a main shaft lumen 21 through which the fluid can flow. A main shaft side distal opening 21a communicating with the main shaft lumen 21 is disposed in the distal end of the main shaft tube 20. The main shaft tube 20 is configured to be flexible enough so that the main shaft tube 20 can indwell the inside of the body after being moderately deformed, and is configured to be rigid enough so that the main shaft tube 20 does not crush the main shaft lumen 21.

In addition, the main shaft tube 20 extends over the above-described insertion planned region 12 and the above-described exposure planned region 14 while maintaining a constant outer diameter and inner diameter. The outer diameter of the main shaft tube 20 can be in a range of 1.3 mm to 5 mm, for example, so that insertion and indwelling of the main shaft tube 20 can be performed on a main pancreatic duct 102 of the pancreas 100. In addition, the diameter (inner diameter of the main shaft tube 20) of the main shaft lumen 21 can be in a range of 1.0 mm to 4.5 mm, for example. The outer diameter and the inner diameter of the main shaft tube 20 may be changed along the axial direction of the main shaft tube 20.

A guide needle 22 is disposed in the proximal portion of the main shaft tube 20 (exposure planned region 14). The guide needle 22 is configured to include a metal material such as aluminum, for example. The guide needle 22 pierces a small hole opened in the jejunum 200 in a pancreaticojejunostomy planned region, and guides the main shaft tube 20 into the jejunum 200. Thereafter, the guide needle 22 pierces outward from the jejunum lumen at a pulling-out planned position of the main shaft tube 20, and the main shaft tube 20 is pulled out of the jejunum 200. Furthermore, the guide needle 22 pierces into a small excision hole at a tube pulling-out position of the abdominal wall, and the main shaft tube 20 is pulled out of the body. The main shaft tube 20 pulled out of the body is cut at a proper position, and the guide needle 22 is cut off. The proximal side of the main shaft tube 20 which is cut off is connected to a drain bag (not illustrated) (including a container which is installed in an aspiration device so as to be capable of applying negative pressure). In addition, a drain port (not illustrated) communicating with the main shaft lumen 21 may be disposed on the distal side from the guide needle 22 of the main shaft tube 20. The drain port drains the fluid outward from the main shaft tube 20, even at atmospheric pressure which does not apply the negative pressure to the main shaft lumen 21.

On the other hand, when the main shaft tube 20 indwells, the insertion planned region 12 of the main shaft tube 20 can be divided into a first site 24 inserted into the pancreas 100, a second site 25 disposed in the boundary portion B of the anastomosis portion In(B), and a third site 26 disposed in the jejunum 200 (including a range where the main shaft tube 20 is removed outward from the jejunum 200 inside the body). The first to third sites 24, 25, and 26 are continuous in order from the distal end to the proximal end of the main shaft tube 20. In addition, the branch tube 30 interlocks with the second site 25.

The main shaft tube 20 may have a fixing mechanism (not illustrated) (for example, a balloon) for fixing the drain tube 10 to the main pancreatic duct 102 or the boundary portion B. In addition, the distal end of the main shaft tube 20 may be formed on a blade surface which is inclined with respect to the shaft center of the main shaft tube 20. In this manner, the main shaft tube 20 is easily inserted into the pancreas 100. Furthermore, the outer peripheral surface of the first site 24 may have a side hole (not illustrated) through which the pancreatic juice is allowed to flow into the main shaft lumen 21.

Furthermore, the main shaft tube 20 can be configured so that the first to third sites 24, 25, and 26 have mutually different hardness. For example, the first and second sites 24 and 25 are hardened compared to the third site 26. In this manner, when the drain tube 10 is removed, it is possible to prevent the first and second sites 24 and 25 from being broken due to the compression of the pancreas 100 or the jejunum 200.

A material configuring the main shaft tube 20 is not particularly limited. However, for example, a resin material can be used. The resin material can include one or more of, but is not limited to, fluorine-based resin such as polytetrafluoroethylene (PTFE), ethylene tetrafluoroethylene copolymer (ETFE), and, perfluoroalkoxy fluorine resin (PFA), olefin-based resin such as polyethylene and polypropylene, or a mixture thereof, polyvinyl chloride, polyurethane, polyester, polyamide, polyether nylon resin, or a mixture of olefin-based resin and ethylene-vinyl acetate copolymer.

On the other hand, eight branch tubes 30 interlock with the outer peripheral surface (the same position in the axial direction) of the second site 25 of the main shaft tube 20. The respective branch tubes 30 may be disposed at equal intervals (at equal angles) along the outer peripheral surface of the main shaft tube 20, and extend radially outward from the main shaft tube 20. In the respective branch tubes 30, the proximal side serves as a fixed end which interlocks with the main shaft tube 20. In contrast, the distal side serves as a free end which is freely displaced with respect to the proximal side.

The shaft center portion of the respective branch tubes 30 has a branch lumen 31 (flow path) communicating with the main shaft lumen 21. In addition, the distal end of the respective branch tubes 30 has a branch side distal opening 31a with which the branch lumen 31 communicates. The diameter (inner diameter of the branch tube 30) of the branch lumen 31 may be in a range 1.0 mm to 3.3 mm, for example.

Furthermore, the respective branch tubes 30 are formed into a tubular body thinner than the main shaft tube 20 to some extent. For example, the outer diameter of the branch tube 30 depends on the outer diameter of the main shaft tube 20 or the number of the branch tubes 30 to be formed. However, the outer diameter can be equal to or smaller than ½ of the outer diameter of the main shaft tube 20.

As illustrated in FIG. 3B, the plurality of branch tubes 30 can be configured so that the axial lengths thereof are alternately changed (different from each other) along the circumferential direction of the main shaft tube 20. That is, a first branch tube group 32 interlocking with the main shaft tube 20 at an interval of 90° is arrayed with a predetermined dimension (for example, approximately equal to the length of the first site 24 of the main shaft tube 20). On the other hand, a second branch tube group 33 interlocking with the main shaft tube 20 while being circumferentially shifted by 45° from the first branch tube group 32 is arrayed with a dimension shorter than that of the first branch tube group 32. For example, the axial length of the second branch tube group 33 may be approximately ¼ to ¾ of the axial length of the first branch tube group 32.

In addition, as illustrated in FIG. 3A, the plurality of branch tubes 30 can be shaped in advance so as not only to face outward in the radial direction of the main shaft tube 20, but also to extend from the intermediate position in the axial direction to the first site 24 side (distal side) of the main shaft tube 20. In this way, the respective branch tubes 30 extend in the distal end direction. Accordingly, it becomes easy to pull the respective branch tubes 30 close to the main shaft tube 20 side when the drain tube 10 is removed, and the drain tube 10 can be easily pulled out. The respective branch tubes 30 may not only interlock with the main shaft tube 20 so that the fixed end protrudes in the direction orthogonal to the main shaft tube 20, but also may obliquely interlock with the main shaft tube 20.

Furthermore, the plurality of branch tubes 30 can be configured to be sufficiently more flexible than the main shaft tube 20. For example, the thickness of the branch tube 30 is made thinner than the main shaft tube 20. Accordingly, it is possible to soften the branch tube 30. In this manner, when the drain tube 10 is removed, each branch tube 30 can be folded so as to easily follow along each side surface of the main shaft tube 20.

A material for configuring the plurality of branch tubes 30 is not particularly limited. However, for example, the resin material used for the above-described main shaft tube 20 may be applicable. In this case, if the main shaft tube 20 and the branch tube 30 are formed of the same material and molded integrally with each other, the drain tube 10 is easily manufactured, and the second site 25 of the main shaft tube 20 is provided with improved rigidity. As a matter of course, the drain tube 10 may be configured so that the main shaft tube 20 and the branch tube 30 are separately manufactured and the branch tube 30 is connected to the outer peripheral surface of the main shaft tube 20 after being manufactured.

Furthermore, the outer peripheral surface of the branch tube 30 may be coated with an adhesion-suppressing layer which suppresses adhesion between the biological organ and the branch tube 30. The adhesion-suppressing layer may be disposed in the main shaft tube 20.

Basically, the drain tube 10 can be configured as described above. Hereinafter, an operation effect thereof will be described.

As described above, the drain tube 10 may be used, for example, for the pancreaticoduodenectomy. In the pancreaticoduodenectomy, for example, as illustrated in FIG. 2A, a portion of the pancreas 100 (pancreatic head side), the duodenum 600, the gall bladder 400, the bile duct 500, and a portion of the jejunum 200 are excised (refer to a two-dot chain line in FIG. 2A). In addition, although FIG. 2A illustrates a procedure for preserving the pylorus ring of the stomach 300, in the pancreaticoduodenectomy, a portion (pylorus side) of the stomach 300 is also excised if necessary.

Then, as illustrated in FIG. 2B, in the reconstructive surgery (end side pancreaticojejunostomy) of the pancreaticoduodenectomy, an end portion of on the exit side (pylorus side) of the stomach 300 is anastomosed with the intermediate position of the jejunum 200. In addition, the cut-off end portion on the side opposite to the bile duct 500 leading to the liver is anastomosed with the intermediate position of the jejunum 200. Furthermore, the pancreas 100 (pancreatic body and pancreatic tail) is anastomosed with the vicinity of the end portion of the excised jejunum 200. The order of the anastomosis may vary in accordance with a selected operation.

After the anastomosis starts to be performed on the pancreas 100 and the jejunum 200, the drain tube 10 according to the present embodiment is caused to indwell across the pancreas 100, the boundary portion B between the pancreas 100 and the jejunum 200[A1]. Specifically, two ports (first port 204 and second port 206) are formed on an intestinal wall 202 so that the drain tube 10 passes through the inside of the jejunum 200. The first port 204 has a function as a pseudo-Valter's papilla, and the distal side from the second site 25 of the main shaft tube 20 passing through the inside of the jejunum 200 is exposed from the jejunum 200. The second port 206 causes the proximal side (third site 26) of the main shaft tube 20 passing through the inside of the jejunum 200 to be exposed from the jejunum 200. The main shaft tube 20 exposed from the second port 206 is taken out of the body from the inside of the body.

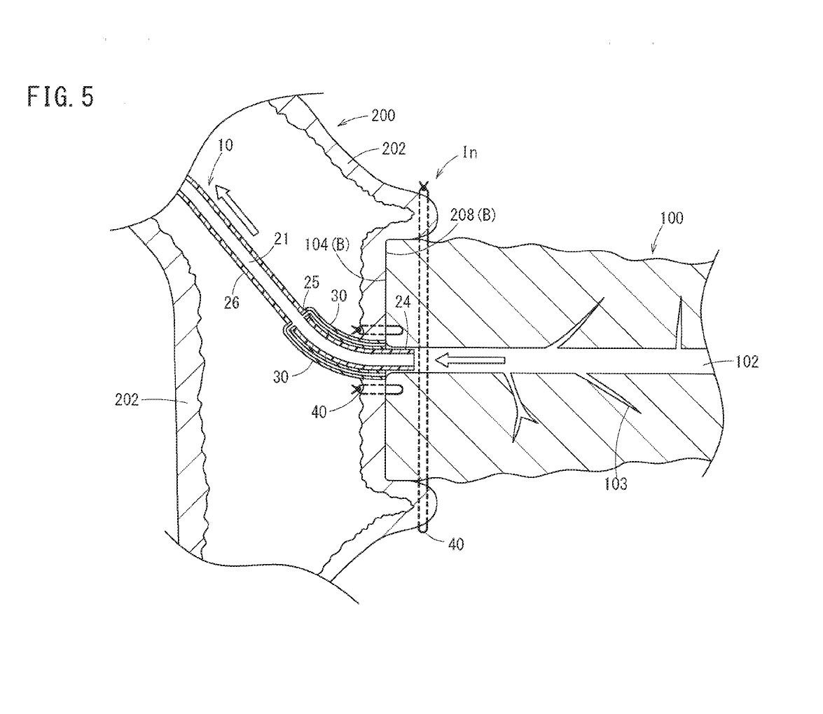

As illustrated in FIG. 5, an operator punctures the first site 24 of the main shaft tube 20, and inserts the main shaft tube 20 into a cut surface 104 of the pancreas 100 before the anastomosis. The first site 24 enters the inside along the main pancreatic duct 102 from the cut surface 104, and the main shaft side distal opening 21a is disposed inside the main pancreatic duct 102. If the first site 24 is substantially inserted into the pancreas 100 and the second site 25 comes close to the cut surface 104, the outer peripheral surface of the branch tube 30 comes into contact with the cut surface 104. At this time, the plurality of branch tubes 30 radially extend outward in the radial direction from the substantially central portion of the cut surface 104 of the pancreas 100 while maintaining mutually different lengths. In this manner, the distal ends (branch side distal opening 31a) are dispersed and disposed in the circumferential direction and the radial direction, respectively.

In this state, the operator disposes the cut surface 104 of the pancreas 100 so as to face and come into surface contact with the intestinal wall 202 around the first port 204 of the jejunum 200 (hereinafter, the outer peripheral surface of the intestinal wall 202 which faces the cut surface 104 is referred to as a facing surface 208). Then, for example, the intestinal wall 202 creates a state where the cut surface 104 of the pancreas 100 and the lateral surrounding connected to the cut surface 104 are covered and reduced in size. The intestinal wall 202 of a portion laterally protruding from the pancreas 100 and the lateral surrounding of the pancreas 100 are sutured using a suture 40. Furthermore, to maintain a contact state between the pancreas 100 and the jejunum 200 and to position the branch tube 30, the anastomosis may be performed using the suture 40 by suturing the branch tube 30, the pancreas 100, and the intestinal wall 202. If the suture 40 is configured so that a biodegradable material is employed and is dissolved inside the body during the indwelling, time and efforts for removal can be saved.

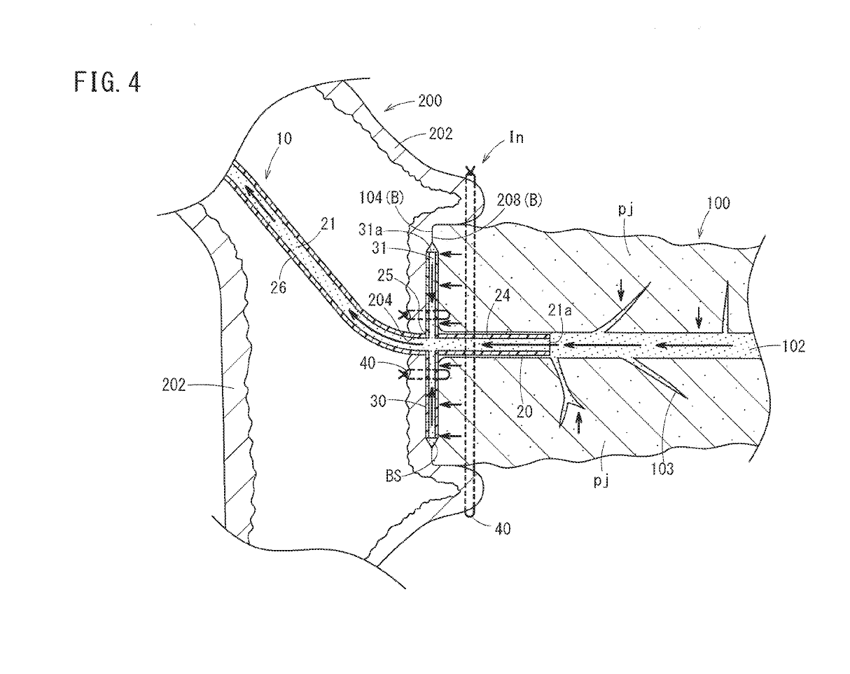

Through the above-described anastomosis, in a state where the branch tube 30 is disposed in the boundary portion B of the anastomosis portion In between the pancreas 100 and the jejunum 200, the drain tube 10 is caused to indwell the inside of a patient. Basically, in the boundary portion B, the cut surface 104 of the pancreas 100 and the facing surface 208 of the jejunum 200 are in contact with each other. However, a slight gap BS (see FIG. 4) is generated between both of these. In addition, after the insertion planned region 12 indwells, the proximal side of the drain tube 10 is connected to the drain bag located outside the body.

In an indwelling state of the drain tube 10, the pancreatic juice (pj) generated by the biological tissue of the pancreas 100 mostly flows from a branch pancreatic duct 103 to the main pancreatic duct 102. Then, the pj flowing in the main pancreatic duct 102 flows into the main shaft lumen 21 from the main shaft side distal opening 21a, and is drained outward from the body through the main shaft lumen 21. In this case, the pj is automatically drained outward from the body by the atmospheric pressure (without applying the negative pressure). However, the pj may be positively aspirated by causing an aspiration device to apply the negative pressure to the main shaft lumen 21.

Then, if the biological tissue in the vicinity of the cut surface 104 generates the pj, the pancreas 100 causes the pj to be exuded from the cut surface 104 rather than causing the pj to flow to the main pancreatic duct 102. That is, a portion of the pj leaks to the gap BS of the boundary portion B between the pancreas 100 and the intestinal wall 202. In contrast, the drain tube 10 can cause the branch tube 30 to collect the pj leaking into the boundary portion B. That is, the branch side distal openings 31a of the plurality of branch tubes 30 are dispersed and disposed in the boundary portion B. Thus, the pj exuded from any position can be fetched by the branch tube 30.

In this case, the pj flows from the branch side distal opening 31a into the branch lumen 31, and flows out to the main shaft lumen 21 through the branch lumen 31. The branch lumen 31 is formed to have a sufficiently small diameter as described above. Accordingly, the pj can be drawn by means of the capillary phenomenon. In addition, if the negative pressure is applied by the aspiration device, it is possible to positively aspirate the pj leaking to the gap BS.

After surgery, in the boundary portion B, the pancreas 100 and the jejunum 200 progressively adhere to each other. In particular, a location of the cut surface 104 of the pancreas 100, which does not face the branch tube 30, is basically in contact with the facing surface 208 of the jejunum 200. Accordingly, the mutual adhesion is promoted. On the other hand, the branch tube 30 forms a radial non-adhesion location between the cut surface 104 and the facing surface 208.

Then, if a certain period of time elapses after the surgery, the drain tube 10 is removed. At this time, the operator can smoothly detach the drain tube 10 from the anastomosis portion In by simply moving the main shaft tube 20 rearward in the proximal end direction.

That is, as illustrated in FIG. 5, when the main shaft tube 20 moves rearward in the proximal end direction, the radially extending branch tube 30 faces inward in the radial direction (the axial direction of the branch tube 30) of the main shaft tube 20. Therefore, the branch tube 30 suppresses the damage of the adhesion portion between the pancreas 100 and the jejunum 200, and is attracted to the first port 204 of the jejunum 200. Then, the branch tube 30 moves from the first port 204 into the jejunum 200. In addition, in the second port 206, the main shaft tube 20 and the branch tube 30 can also be similarly pulled out, and the drain tube 10 is satisfactorily taken out of the body. When the drain tube 10 is removed, the branch tube 30 may be folded to the main shaft tube 20 side, and the branch lumen 31 may be crushed. After the branch tube 30 is detached, the pancreas 100 and the jejunum 200 start adhesion in a location where the branch tube 30 is disposed.

As described above, in the drain tube 10 (medical tube 10), the distal end of the branch tube 30 interlocking with the main shaft tube 20 is disposed in the boundary portion B between the pancreas 100 and the jejunum 200. Accordingly, it is possible to collect the pj of the boundary portion B. That is, the pj of the boundary portion B flows into the branch lumen 31 of the branch tube 30, and flows to the main shaft lumen 21 of the main shaft tube 20. Accordingly, the drain tube 10 not only can cause the main shaft tube 20 inserted into the biological organ to drain the pj, but also can cause the branch tube 30 to satisfactorily drain the pj of the boundary portion B where the pancreas 100 and the jejunum 200 are anastomosed with each other. As a result, it is possible to encourage recovery of a patient by suppressing the inconvenience that the pj may flow out into the patient's body.

In this case, the branch tube 30 is configured to be more flexible than the main shaft tube 20. Accordingly, the branch tube 30 can indwell to easily follow along the shape of the boundary portion B between the pancreas 100 and the jejunum 200. In addition, when the drain tube 10 is removed, the branch tube 30 moves along the arrangement position of the branch tube 30 which is the non-adhesion location. Accordingly, while suppressing the damage of the adhesion location between the pancreas 100 and the jejunum 200, the branch tube 30 reaches the main shaft tube 20. Accordingly, the branch tube 30 moves integrally with the main shaft tube 20, and can be smoothly detached from the boundary portion B. Furthermore, the outer diameter of the branch tube 30 is smaller than the outer diameter of the main shaft tube 20. Accordingly, the gap BS where the branch tube 30 is disposed decreases, and the adhesion between the pancreas 100 and the jejunum 200 can be promoted. When the drain tube 10 is removed, the above-described configuration enables the branch tube 30 to smoothly move rearward from the boundary portion B.

Here, the branch tube 30 is shaped in advance so as to face outward in the radial direction of the main shaft tube 20 and toward the distal side. Accordingly, when the branch tube 30 is disposed, the distal end of the branch tube 30 can be easily disposed at a position separated from the main shaft tube 20. Then, the plurality of branch tubes 30 extend radially outward in the radial direction of the main shaft tube 20. Accordingly, the distal ends of the respective branch tubes 30 can be disposed in the vicinity of the peripheral edge of the boundary portion B so as to be separated from each other in the circumferential direction. Accordingly, the pj of the boundary portion B can be more reliably and effectively collected. In addition, the branch tubes 30 adjacent to each other have mutually different axial lengths. Accordingly, the pj can be collected by disposing the distal end of the branch tube 30 at a relatively close position of the main shaft tube 20.

The medical tube 10 is not limited to the above-described configuration, and various configurations can be adopted. For example, the outer peripheral surface of the branch tube 30 may have one or more lateral ports (not illustrated) communicating with the branch lumen 31. In this manner, the fluid can flow into the branch lumen 31 from the lateral port, and thus, the collection efficiency of the pj can be improved. The branch tube 30 may not include the branch side distal opening 31a, and may include only the lateral port.

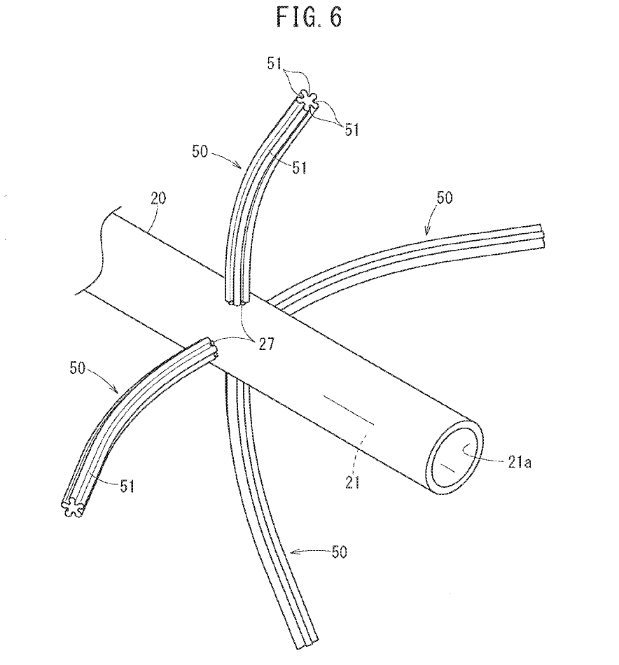

The drain tube 10, as illustrated in FIG. 6, can be different from the drain tube 10 in that a plurality of branch tubes 50 have a groove portion 51, instead of the branch tube 30 having the branch lumen 31.

More specifically, the branch tube 50 includes a plurality of the groove portions 51 at different positions in the circumferential direction, in a cross section perpendicular to the shaft center of the branch tube 50. The respective groove portions 51 extend from the distal end of the branch tube 50 to the proximal end interlocking with the main shaft tube 20. The respective groove portions 51 open the outer peripheral surface side (outward in the radial direction) of the branch tube 50, and the fluid can flow into the groove portion 51 at any position in the axial direction.

In addition, the respective branch tubes 50 interlock with the outer peripheral surface of the second site 25 of the main shaft tube 20. The outer peripheral surface of the second site 25 has a communicating port 27 for communicating with the main shaft lumen 21 at each position facing the groove portion 51 of the respective branch tubes 30. In this manner, the fluid flowing through the groove portion 51 flows into the main shaft lumen 21 via the communicating port 27.

As described above, even in the configuration where the respective branch tubes 50 have the plurality of groove portions 51, the branch tubes 50 are disposed in the boundary portion B between the pancreas 100 and the jejunum 200. In this manner, the pj can be satisfactorily collected. In particular, in a state where the branch tube 50 including the plurality of groove portions 51 is interposed between the cut surface 104 of the pancreas 100 and the facing surface of the jejunum 200, the pj is allowed to flow into the groove portion 51 from any position in the radial direction of the cut surface 104. Accordingly, the pj can smoothly flow to the main shaft lumen 21. In addition, the branch tube 50 suppresses adhesion with the biological tissue by repeatedly forming irregularities in the circumferential direction of the outer peripheral surface. When the drain tube 10 is removed, the branch tube 50 can smoothly move in the radial direction (to the main shaft tube 20 side). The groove portion 51 may also be applicable to other examples of modifications (to be described later).

As illustrated in FIG. 7A, a plurality of branch tubes 55 can be different from the above-described branch tubes 30 and 50 in that the branch tubes 55 have a terminal branch tube 56 which further branches from the intermediate position in the axial direction. One or more terminal branch tubes 56 are disposed in the axial direction of the branch tube 55, and obliquely protrude toward the distal end (free end) of the branch tube 55 as illustrated in FIG. 7A. The terminal branch tube 56 may obliquely protrude in the direction orthogonal to the axial direction of the branch tube 55 or toward the proximal end (fixed end).

The terminal branch tube 56 internally has a terminal branch lumen 57 which allows communication between a terminal branch side distal opening 57a disposed in the distal end of the terminal branch tube 56 and a branch lumen 58 of the branch tube 55. Therefore, similarly to the branch tube 30, the terminal branch tube 56 can collect the fluid from the terminal branch side distal opening 57a via the terminal branch lumen 57.

In addition, the terminal branch tube 56 can be configured to be more flexible than the branch tube 55. In this manner, when the drain tube 10 is removed, if a rearward movement force is applied from the branch tube 55 to the terminal branch tube 56, the terminal branch tube 56 easily reaches the branch tube 55 side. Accordingly, smooth rearward movement can be realized by suppressing the resistance applied to the terminal branch tube 56.

As illustrated in FIG. 7B, a plurality of branch tubes 60 may be different from the above-described branch tubes 30, 50, and 55 in that the branch tube 60 is further divided into two in the distal end (first branch end portion 61) in the extending direction. In addition, the dually divided branch tube 60 is divided into two in the distal end (second branch end portion 62) in the extending direction, and is further divided into two in the distal end (third branch end portion 63) in the extending direction.

The respectively divided branch tubes 60 internally have a branch lumen 64, into which the fluid can flow from each distal end of the divided portion. In this manner, the fluid can be satisfactorily collected. The distal side tube of the branch tube 60 is configured to be more flexible than the first to third branch end portions 61, 62, and 63. Accordingly, the branch tube 60 can be more smoothly pulled out. In addition, the branch tube 60 may be divided into three or more, or may be configured to have two, four, or more branch end portions.

As illustrated in FIG. 7C, a branch tube 65 can also be different from the above-described branch tubes 30, 50, 55, and 60 in that one branch tube 65 extends from the main shaft tube 20 and has a spiral shape in a plan view when viewed in the axial direction of the main shaft tube 20. In this case, the branch tube 65 is formed in a spiral shape in advance. In addition, the outer peripheral surface of the branch tube 65 has a plurality of lateral ports (not illustrated) communicating with a branch lumen 66. The lateral port enables the fluid to flow from the intermediate position in the axial direction to the branch lumen 66.

The branch tube 65 configured as described above is spirally disposed in the boundary portion B between the pancreas 100 and the jejunum 200. Accordingly, the pj can be fetched at every position of the boundary portion B. In addition, when the drain tube 10 is removed, if the rearward movement force is applied from the main shaft tube 20 to the branch tube 65, the branch tube 65 is moved along the spiral shape. Accordingly, the branch tube 65 can be smoothly pulled out by suppressing the damage of the adhesion portion between the biological organs.

As illustrated in FIG. 7D, a branch tube 70 can be different from the above-described branch tubes 30, 50, 55, 60, and 65 in that a plurality of branch tubes 70 extend from the main shaft tube 20 and has an arc shape in a plan view when viewed in the axial direction of the main shaft tube 20. The branch tube 70 internally includes a branch lumen 71, and has a branch side distal opening 71a and a lateral port (not illustrated). Accordingly, the pj can be fetched. In addition, the branch tube 70 formed in an arc shape rotates the main shaft tube 20 when the drain tube 10 is removed. In this manner, the branch tube 70 can reach the main shaft tube 20 by being moved in the rotation direction. Therefore, the branch tube 70 can be more smoothly pulled out.

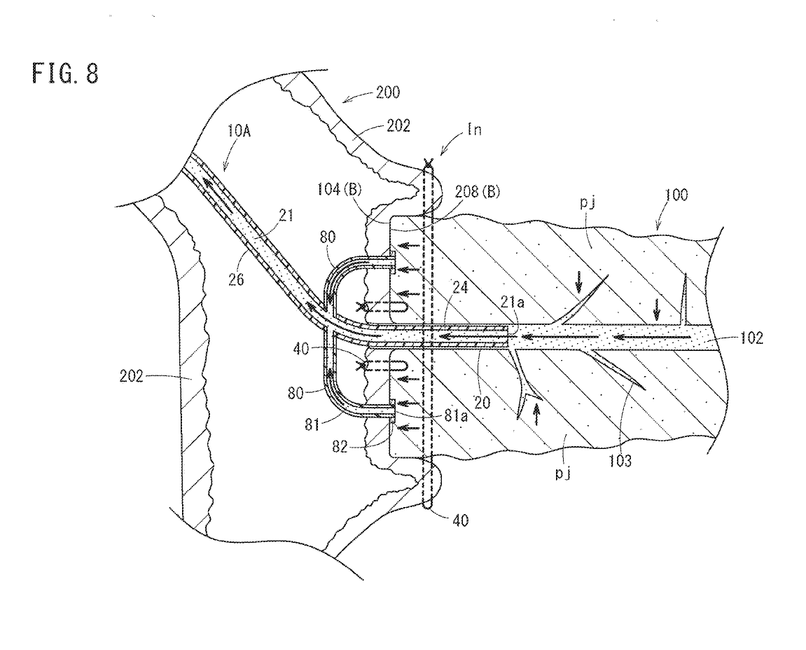

As illustrated in FIG. 8, another example of a drain tube 10A (medical tube 10A) is different from the above-described drain tube 10 in that a branch tube 80 is caused to extend from the inside of the jejunum 200 and only the distal side is disposed in the boundary portion B between the pancreas 100 and the jejunum 200. Therefore, the main shaft tube 20 is configured so that the first site 24 is long. The first site 24 is inserted from the inside of the jejunum 200 into the main pancreatic duct 102 of the pancreas 100 after penetrating through the intestinal wall 202.

On the other hand, the branch tube 80 is formed to be longer than the axial length of the first site 24 from the interlocking portion (second site 25) of the main shaft tube 20. In this manner, the branch tube 80 can be curved outward (radially) in the radial direction so as to extend to the distal side. In this manner, the distal end of the branch tube 80 indwells the boundary portion B so that a branch side distal opening 81a faces the cut surface 104 of the pancreas 100 at a position separated from the main shaft tube 20, thereby allowing the pj leaking to the boundary portion B to flow into a branch lumen 81.

In addition, to prevent the distal end of the branch tube 80 from slipping out of the intestinal wall 202 or to prevent the distal end from puncturing the pancreas 100, the distal side of the branch tube 80 has distal fixing means 82. For example, the distal fixing means 82 includes a structure in which the distal end of the branch tube 80 is sutured to the intestinal wall 202 by using the suture 40. In addition, for example, the distal fixing means 82 may have a structure in which the distal end of the branch tube 80 is torn and bent outward in the radial direction. Alternatively, a structure may be employed in which a balloon (inflating/deflating structure), a flange, or a hook is disposed in the distal end of the branch tube 80.

The drain tube 10A configured as described above can obtain an advantageous effect the same as that of the drain tube 10 described previously. In particular, according to the drain tube 10A, the branch tube 80 branches from the main shaft tube 20 inside the jejunum 200, and only the distal side is disposed in the boundary portion B. Accordingly, it is possible to minimize a range where the branch tube 80 hinders the adhesion between the biological organs. In addition, when the drain tube 10A is removed, the branch tube 80 is pulled out in the proximal end direction. In this manner, the distal end of the branch tube 80 can smoothly move rearward from the boundary portion B.

Furthermore, the drain tube 10A includes the distal fixing means 82. Accordingly, the distal end of the branch tube 80 can be prevented from slipping out of the boundary portion B, and the pj of the boundary portion B can be more satisfactorily collected. The distal fixing means 82 may be disposed in the branch tube 30 of the drain tube 10.

Alternatively, another configuration of the drain tubes 10 and 10A, as described hereinafter, may be adopted. A material (biocompatible material), which is soluble when disposed inside the living body for a long time, may be applied to the branch tube 30, and may be separated from the main shaft tube 20 when the drain tubes 10 and 10A are removed. In this manner, while the shape of the branch tube 30 is continuously maintained without being melted, the pj can be satisfactorily collected. In contrast, after the branch tube 30 is melted, the main shaft tube 20 can be easily pulled out.

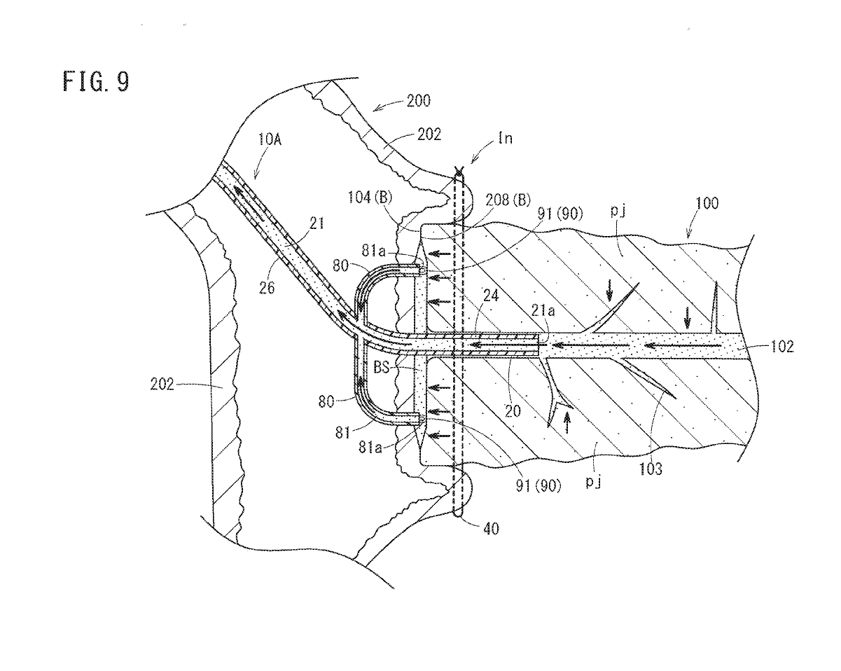

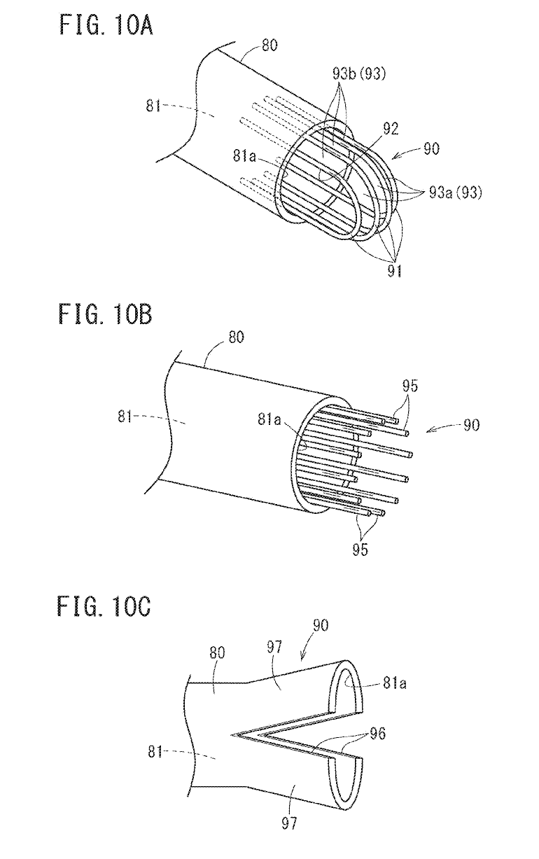

In addition, in the drain tube 10A as illustrated in FIG. 9, the distal end of the branch tube 80 can have a structure (distal opening support portion 90) for allowing the pj to stably flow to the branch lumen 81. As illustrated in FIG. 10A, the distal opening support portion 90 is configured to include a plurality of (four) the support wires 91. The four support wires 91 are juxtaposed along the width direction (direction orthogonal to the axial direction of the branch tube 80) of the branch tube 80. The respective support wires 91 extend so as to be short from the distal end to the distal side of the branch tube 80, and are formed in an arch shape which is folded back into an arc in the distal end. The respective support wires 91 are firmly fixed to the inner peripheral surface in the vicinity of a branch side distal opening 81a of the branch tube 80 by fixing means such as welding or adhesion.

The respective support wires 91 protrude in an arch shape, and form a lateral opening portion 92 which internally communicates with the branch side distal opening 81a. In addition, the four support wires 91 are fixed to each other while leaving a clearance therebetween to some degree (that is, in a non-contact manner). In this manner, a gap 93 into which the pj can flow is formed between the support wires 91. In the gap 93, a forward opening portion 93a is formed in the distal end of the distal opening support portion 90, and lateral opening portion 93b is formed laterally (direction orthogonal to the lateral opening portion 92) in the distal opening support portion 90.

As illustrated in FIGS. 9 and 10A through 10C, the drain tube 10, configured as described above, indwells the inside of the body by disposing the distal end of the branch tube 80 in the boundary portion B, when in use. In this case, the distal opening support portion 90 prevents the branch side distal opening 81a from being closed by the pancreas 100 in such a way that the four support wires 91 come into contact with and support the pancreas 100. In addition, the lateral opening portions 92 and 93b and the forward opening portion 93a of the distal opening support portion 90 allow the pj to pass therethrough. Accordingly, the pj is allowed to satisfactorily flow to the branch lumen 81 of the branch tube 80. Therefore, the pj of the boundary portion B can be effectively drained.

The distal opening support portion 90 may employ various structures. For example, as illustrated in FIG. 10B, a configuration may be adopted in which a plurality of (12 in FIG. 10B) elongated linear protruding bodies 95 are disposed in the branch side distal opening 81a of the branch tube 80. In addition, for example, as illustrated in FIG. 10C, a configuration may be adopted in which a pair of slits 96 is cut from the distal end of the branch tube 80 and the distal end of the branch tube 80 is divided into a pair of semi-cylindrical portions 97.

The present invention not limited to the above embodiments. As a matter of course, various modifications can be made without departing from the gist of the present invention. For example, the medical tubes 10 and 10A are applicable not only to a portion where two biological organs are anastomosed with each other, but also to a portion where a plurality of biological organs are connected each other using other connection methods. In addition, the medical tubes 10 and 10A may also be used to cause the fluid to flow out to the connection section of the biological organ by supplying the fluid from the proximal side.

### DESCRIPT ION OF REFERENCE NUMERALS AND SIGNS

10, 10A: medical tube (drain tube)

20: main shaft tube

21: main shaft lumen

21a: main shaft side distal opening

24: first site

25: second site

26: third site

30, 50, 55, 60, 65, 70, 80: branch tube

31, 58, 64, 66, 71, 81: branch lumen

31a, 71a, 81a: branch side distal opening

51: groove portion

82: distal fixing means

100: pancreas

104: cut surface

200: jejunum

202: intestinal wall

208: facing surface

B: boundary portion

In: anastomosis portion

pj: pancreatic juice

1. A medical tube comprising:
an elongated main shaft tube that internally has a main shaft lumen; and
at least one branch extending portion that extends from an intermediate position in an axial direction of the elongated main shaft tube, wherein the branch extending portion has a flow path which communicates with the main shaft lumen from the outside of the elongated main shaft tube, wherein the elongated main shaft tube is inserted into a first biological organ, and wherein the branch extending portion can be inserted into a portion between the first biological organ and a second biological organ.

2. The medical tube according to claim 1, wherein the flow path is a branch lumen which is disposed inside the branch extending portion, and through which an opening, disposed in a distal end or on a side surface of the branch extending portion, and the main shaft lumen communicate with each other.

3. The medical tube according to claim 1, wherein the flow path is a groove portion which opens a side surface of the branch extending portion, and which extends along the axial direction of the branch extending portion.

4. The medical tube according to any one of claims 2, wherein the branch extending portion is configured to be more flexible than the elongated main shaft tube.

5. The medical tube according to any one of claims 4, wherein an outer diameter of the branch extending portion is smaller than an outer diameter of the elongated main shaft tube.

6. The medical tube according to any one of claims 5, wherein the branch extending portion is shaped so as to face outward in a radial direction of the main shaft tube, and toward a distal side of the main shaft tube.

7. The medical tube according to any one of claims 6, wherein a plurality of the branch extending portions interlock with each other at a same position in the axial direction of the main shaft tube, and extend radially outward in the radial direction of the main shaft tube.

8. The medical tube according to of claim 7, wherein the plurality of the branch extending portions comprises a first branch extending portion and a second branch extending portion, wherein the first branch extending portion is adjacent to the second branch extending portion, and wherein the first branch extending portion has a different axial length than the second branch extending portions.

9. The medical tube according to any one of claims 8, wherein a distal portion of the branch extending portion includes distal fixing means for fixing the branch extending portion to the second biological organ.

10. The medical tube according to any one of claims 9, wherein the distal end of the branch extending portion has a distal opening support portion which can support the first or second biological organ, and wherein the distal opening support portion has a forward opening portion which communicates with the branch lumen, and which is open in a distal end direction of the branch extending portion, and a lateral opening portion which communicates with the branch lumen, and which is open in a lateral portion of the branch extending portion.

11. A medical tube comprising:
a main shaft tube comprising a main shaft lumen formed internal to the main shaft tube, wherein the main shaft tube is inserted into a first biological organ; and
a first branch extending portion between a proximal side and a distal side of the main shaft tube, wherein the first branch extending portion extends in an axial direction from the main shaft tube, wherein the first branch extending portion has a flow path which communicates with the main shaft lumen from an outside of the elongated main shaft tube, and wherein the first branch extending portion can be inserted into a portion between the first biological organ and a second biological organ.

12. The medical tube according to claim 11, wherein the flow path is one of:
a branch lumen disposed internal to the branch extending portion, and through which an opening, disposed in a distal end or on a side surface of the branch extending portion, and the main shaft lumen communicate with each other; or
a groove portion which opens a side surface of the branch extending portion, and which extends along the axial direction of the branch extending portion and is aligned with a second opening the main shaft tube.

13. The medical tube according to any one of claims 11, wherein the branch extending portion is configured to be more flexible than the elongated main shaft tube, wherein an outer diameter of the branch extending portion is smaller than an outer diameter of the elongated main shaft tube, and wherein the branch extending portion is shaped so as to face outward in a radial direction of the main shaft tube and bend toward a distal side of the main shaft tube.

14. The medical tube according to any one of claims 11, wherein the first branch extending portion is divided into two or more distal ends extending radially from the first branch extending portion.

15. The medical tube according to of claim 11, wherein the first branch extending portion has a spiral shape that is shaped circumferentially around the main shaft tube.

16. A medical tube comprising:
a main shaft tube comprising a main shaft lumen formed internal to the main shaft tube, wherein the main shaft tube is inserted into a first biological organ; and
a first branch extending portion between a proximal side and a distal side of the main shaft tube, wherein the first branch extending portion extends in an axial direction from the main shaft tube, wherein the first branch extending portion has a first flow path which communicates with the main shaft lumen from an outside of the elongated main shaft tube, and wherein the first branch extending portion can be inserted at a first location into a portion between the first biological organ and a second biological organ.

17. The medical tube according to claim 16, further comprising a second branch extending portion between the proximal side and the distal side of the main shaft tube, wherein the second branch extending portion extends in an axial direction from the main shaft tube, wherein the second branch extending portion has a flow path which communicates with the main shaft lumen from an outside of the elongated main shaft tube, and wherein the second branch extending portion can be inserted at a second location into the portion between the first biological organ and the second biological organ.

18. The medical tube according to any one of claims 17, wherein the first branch extending portion and the second branch extending portion interlock with each other at a same position in the axial direction of the main shaft tube.

19. The medical tube according to any one of claims 18, wherein the first branch extending portion is adjacent to the second branch extending portion, and wherein the first branch extending portion has a different axial length than the second branch extending portions.

20. The medical tube according to of claim 18, wherein the first branch extending portion and the second branch extending portion each have, at least partially, a spiral shape that is shaped circumferentially around the main shaft tube.
